SEQUENCE LISTING (44 sequences)

SEQ ID NO: 1 - DNA, 25 bp, Artificial
  Description of Artificial Sequence Oligonucleotide to act as a primer for PCR
gcaagacatgctagtaccacaggag

SEQ ID NO: 2 - DNA, 21 bp, Artificial
  Description of Artificial Sequence Oligonucleotide to act as a primer for PCR
ctgctgacccagatgcagttc

SEQ ID NO: 3 - DNA, 24 bp, Artificial
  Description of Artificial Sequence Oligonucleotide to act as a primer for PCR
catgagttcacatcagcagaagca

SEQ ID NO: 4 - DNA, 21 bp, Artificial
  Description of Artificial Sequence Oligonucleotide to act as a primer for PCR
ggctcaggaaccttggtgtca

SEQ ID NO: 5 - DNA, 23 bp, Artificial
  Description of Artificial Sequence Oligonucleotide to act as a primer for PCR
ggatccacctgtctctgctcaag

SEQ ID NO: 6 - DNA, 20 bp, Artificial
  Description of Artificial Sequence Oligonucleotide to act as a primer for PCR
ttccaccctggcacatcaag

SEQ ID NO: 7 - DNA, 21 bp, Artificial
  Description of Artificial Sequence Oligonucleotide to act as a primer for PCR
ccagcagaagtgcccacctaa

SEQ ID NO: 8 - DNA, 24 bp, Artificial
  Description of Artificial Sequence Oligonucleotide to act as a primer for PCR
gggtgttgctttggaagtacatga

SEQ ID NO: 9 - DNA, 20 bp, Artificial
  Description of Artificial Sequence Oligonucleotide to act as a primer for PCR
cagcagtcatcgggacaagg

SEQ ID NO: 10 - DNA, 24 bp, Artificial
  Description of Artificial Sequence Oligonucleotide to act as primer for PCR
attacaggcaaatgggagtgacag

SEQ ID NO: 11 - DNA, 25 bp, Artificial
  Description of Artificial Sequence Oligonucleotide to act as a primer for PCR
ctgaggcttcagcaacaatgtctta

SEQ ID NO: 12 - DNA, 20 bp, Artificial
  Description of Artificial Sequence Oligonucleotide to act as a primer for PCR
aaggctcagggcactttgga

SEQ ID NO: 13 - DNA, 22 bp, Artificial
  Description of Artificial Sequence Oligonucleotide to act as a primer for PCR
atcctgagacttcagaaagatg

SEQ ID NO: 14 - DNA, 24 bp, Artificial
  Description of Artificial Sequence Oligonucleotide to act as a primer for PCR
ttgttctgaagattgctgccatgc

SEQ ID NO: 15 - DNA, 24 bp, Artificial
  Description of Artificial Sequence Oligonucleotide to act as a primer for PCR
catgtgcctgtgataactcctgaa

SEQ ID NO: 16 - DNA, 23 bp, Artificial
  Description of Artificial Sequence Oligonucleotide to act as a primer for PCR
tctgagcatctgggacagtgaag

SEQ ID NO: 17 - DNA, 22 bp, Artificial
  Description of Artificial Sequence Oligonucleotide to act as a primer for PCR
ctgtgaccgatacgggattgtg

SEQ ID NO: 18 - DNA, 22 bp, Artificial
  Description of Artificial Sequence Oligonucleotide to act as a primer for PCR
acctctaggtggtgccgaagtg

SEQ ID NO: 19 - DNA, 22 bp, Artificial
  Description of Artificial Sequence Oligonucleotide to act as a primer for PCR
ggactgcctgagcaagaatgtg

SEQ ID NO: 20 - DNA, 20 bp, Artificial
  Description of Artificial Sequence Oligonucleotide to act as a primer for PCR
taagctgctgctctgggttt

SEQ ID NO: 21 - DNA, 20 bp, Artificial
  Description of Artificial Sequence Oligonucleotide to act as a primer for PCR
acacagcccattctgctccg

SEQ ID NO: 22 - DNA, 21 bp, Artificial
  Description of Artificial Sequence Oligonucleotide to act as a primer for PCR
tgcaaaggagcgattatgatt

SEQ ID NO: 23 - DNA, 21 bp, Artificial
  Description of Artificial Sequence Oligonucleotide to act as a primer for PCR
gggaccatacagagtattcct

SEQ ID NO: 24 - DNA, 21 bp, Artificial
  Description of Artificial Sequence Oligonucleotide to act as a primer for PCR
ggtactaagaccttcagcttc

SEQ ID NO: 25 - DNA, 20 bp, Artificial
  Description of Artificial Sequence Oligonucleotide to act as a primer for PCR
tatttggctcacctcgttcc

SEQ ID NO: 26 - DNA, 20 bp, Artificial
  Description of Artificial Sequence Oligonucleotide to act as a primer for PCR
ccaggacttcctttgctcag

SEQ ID NO: 27 - DNA, 23 bp, Artificial
  Description of Artificial Sequence Oligonucleotide to act as a primer for PCR
agcttcaggattcatcaggagca

SEQ ID NO: 28 - DNA, 21 bp, Artificial
  Description of Artificial Sequence Oligonucleotide to act as a primer for PCR
tgggcagatcacaggctaagg

SEQ ID NO: 29 - DNA, 22 bp, Artificial
  Description of Artificial Sequence Oligonucleotide to act as a primer for PCR
tacttgagcaccgatctgcttc

SEQ ID NO: 30 - DNA, 21 bp, Artificial
  Description of Artificial Sequence Oligonucleotide to act as a primer for PCR
gagaagctctgaaaggccggt

SEQ ID NO: 31 - DNA, 23 bp, Artificial
  Description of Artificial Sequence Oligonucleotide to act as a primer for PCR
gagctaagaaaaggaagtcctca

SEQ ID NO: 32 - DNA, 24 bp, Artificial
  Description of Artificial Sequence Oligonucleotide to act as a primer for PCR
ttattcagggagtgaacgataaat

SEQ ID NO: 33 - DNA, 22 bp, Artificial
  Description of Artificial Sequence Oligonucleotide to act as a primer for PCR
agcctaccatggatacacagtt

SEQ ID NO: 34 - DNA, 27 bp, Artificial
  Description of Artificial Sequence Oligonucleotide to act as a primer for PCR
gacacagaaaacatcaacagcattttt

SEQ ID NO: 35 - DNA, 20 bp, Artificial
  Description of Artificial Sequence Oligonucleotide to act as a primer for PCR
tggctcacctcgttccacag

SEQ ID NO: 36 - DNA, 21 bp, Artificial
  Description of Artificial Sequence Oligonucleotide to act as a primer for PCR
caggccgatcacaggctaaga

SEQ ID NO: 37 - DNA, 23 bp, Artificial
  Description of Artificial Sequence Oligonucleotide to act as a primer for PCR
gccattcagttgtagatctttga

SEQ ID NO: 38 - DNA, 21 bp, Artificial
  Description of Artificial Sequence Oligonucleotide to act as a primer for PCR
gcgagattaggcaatgatctg

SEQ ID NO: 39 - DNA, 20 bp, Artificial
  Description of Artificial Sequence Oligonucleotide to act as a primer for PCR
ttccacaacctggaaacaca

SEQ ID NO: 40 - DNA, 20 bp, Artificial
  Description of Artificial Sequence Oligonucleotide to act as a primer for PCR
ttcagggaccttggtgtagc

SEQ ID NO: 41 - DNA, 20 bp, Artificial
  Description of Artificial Sequence Oligonucleotide to act as a primer for PCR
caagtgaagcagccttgtca

SEQ ID NO: 42 - DNA, 20 bp, Artificial
  Description of Artificial Sequence Oligonucleotide to act as a primer for PCR
atctggtagccaggatggtg

SEQ ID NO: 43 - DNA, 21 bp, Artificial
  Description of Artificial Sequence Oligonucleotide to act as a primer for PCR
gccaaggtcatccatgacaac

SEQ ID NO: 44 - DNA, 21 bp, Artificial
  Description of Artificial Sequence Oligonucleotide to act as a primer for PCR
gtccaccaccctgttgctgta

(19) United States
(12) Patent Application Publication
(10) Pub. No.: US 2012/0100222 A1
(43) Pub. Date: Apr. 26, 2012

(54) SKIN FUNCTION-IMPROVING COMPOSITION

(75) Inventors: Hidetsugu Goto, Hokkaido (JP); Keiko Akashi, Hokkaido (JP); Toshihiro Tsuji, Hokkaido (JP); Mitsunori Hashimoto, Hokkaido (JP); Masaki Yamani, Hokkaido (JP); Hidemasa Motoshima, Hokkaido (JP)

(73) Assignee: YOTSUBA MILK PRODUCTS CO., LTD.,, Hokkaido (JP)

(21) Appl. No.: 13/319,181

(22) PCT Filed: Mar. 26, 2010
(86) PCT No.: PCT/JP2010/055354

§ 371 Date: Nov. 7, 2011

(30) Foreign Application Priority Data
May 20, 2009 (JP) 2009-121578

(51) Int. Cl. A61K 35/20; A61P 17/00

(52) U.S. Cl. 424/535

(57) ABSTRACT

A skin function-improving composition for oral ingestion, which contains a milk-fat globule membrane. The composition is inexpensive, is highly safe, can be ingested orally, and has an activity of improving the dry condition of the skin so as to moisturize the skin. In particular, the milk-fat globule membrane is obtained from buttermilk or a processed product thereof. In particular, the composition has an activity of reducing transepidermal water loss or an activity of suppressing epidermal thickness. The composition can be ingested in the form of a food or beverage.

### TECHNICAL FIELD

The present invention relates to a skin function-improving composition, and specifically, to a skin function-improving composition for oral ingestion which contains a milk fat globule membrane isolated from milk as an active ingredient and is highly safe, in particular, a skin function-improving composition having an activity of improving a dry condition of a skin.

### BACKGROUND ART

Skin is the largest organ in the human body, which protects life actions in the body from a variety of environments. Water accounts for about 70% in the human body, and hence avoidance of an excess loss of water from the body is given as one role of the skin.

In the case where the amount of water in an external environment is small, a large amount of water is lost from the skin, resulting in a state of so-called dry skin. Further, in recent years, because of excessive use of face wash or soap by excess cleanliness, the number of persons suffering from loss of a sebum component and dry skin caused by the loss has been increasing.

The dry skin is known to cause not only itch or rhagades but also an increase in trans epidermal water loss (TEWL) of the skin because a water retention ability in the body, which is a major function of the skin, has been lost. The skin having an increased TEWL has a lowered function to protect the skin from an external environment, which may cause a disease such as bacterial infection. If the TEWL is lowered, that is, if an excessive water loss from the skin is suppressed to improve the water retention ability of the skin, it is possible to keep the skin healthy.

Meanwhile, skin thickness also disturbs the normal water retention ability, resulting in an increase in the TEWL. That is, suppression of the skin thickness can normalize the water retention ability of the skin to keep healthy skin.

In addition, a layer which has contact with the environment in skin tissues is called horny layer, and includes flat corneocytes formed by denucleation of epidermal keratinocytes and intercellular lipids. The cell membranes of corneocytes form a strong and hydrophobic horny layer supported by a cornified cell envelope and forms a basis of a horny layer barrier function.

It has been found that, in the abnormal skin of a mouse, expression of genes encoding small proline-rich proteins which are components of the cornified cell envelope (Sprr genes) extremely varies (Non Patent Literature 1).

From the fact that the horny layer is formed in a complex manner, it is estimated that a variation in expression of a specific gene provides an inhibitory effect on normal growth of the horny layer. That is, if an abnormal variation in gene expression in abnormality in the skin can be recovered to a normal expression level, the horny layer may be formed normally to improve the abnormality in the skin.

Conventionally, as disclosed in Patent Literature 1, in order to suppress the TEWL, an external preparation for skin application or the like has been developed, but in some cases, the preparation causes insufficient absorption of a medicinal component to the inside of the skin or exhibits an inflammatory reaction to a chemical substance other than the medicinal component in the skin injured due to dry skin. Further, the preparation has many problems in usability because of sensory avoidance due to its high stickiness of the external preparation and poor stability in the skin.

In order to avoid such problems, as shown in Patent Literatures 2 to 4, in recent years, many foods for decreasing the TEWL have been developed. However, the foods are not satisfactory because the mechanisms are unknown, the flavors are not good as foods, and the foods are very expensive as foods.

On the other hand, a fat in milk is coated with a milk fat globule membrane and is dispersed in milk as a fat globule. The milk fat globule membrane is formed of characteristic membrane proteins and lipids, and contributes to stabilization of the fat in milk.

It should be noted that the milk fat globule membrane is usually abbreviated as MFGM in many cases, but in recent years, the membrane is also referred to as a milk lipid globule membrane and may be abbreviated as MLGM. Here, the term of milk fat globule membrane is used and abbreviated as MFGM.

As an industrial method for concentration of the MFGM, the method disclosed in Patent Literature 5 is known. It has been reported by Kanno that the MFGM has high emulsifiability (Non Patent Literature 2), but development of application other than the emulsifiability has been demanded.

### CITATION LIST

### Patent Literature

[Patent Literature 1] JP 2008-56570 A
[Patent Literature 2] JP 2007-306882 A
[Patent Literature 3] JP 2004-315512 A
[Patent Literature 4] JP 2004-238285 A
[Patent Literature 5] JP 09-172962 A

### Non Patent Literature

[Non Patent Literature 1] Songmei Geng, J. Cell. Sci. 119(23), 4901-4912, (2006)
[Non Patent Literature 2] Kanno, C. J. Food. Sci. 54(6), 1534-1539, (1989)

### SUMMARY OF INVENTION

### Technical Problem

An object of the present invention is to provide a skin function-improving composition ‘which is inexpensive and highly safe and can be orally ingested’ and ‘which has en activity of improving a dry condition of the skin (in particular, has an activity of reducing a TEWL and an activity of suppressing skin thickness to keep moisturizing the skin).’

### Solution to Problem

The inventors of the present invention have made intensive studies to solve the above-mentioned conventional problems, and as a result, have found that oral ingestion of the MFGM separated from milk (in particular, buttermilk or a processed product thereof) by microfiltration can reduce a TEWL in the skin and skin thickness and improve a dry condition of the skin.

Further, the inventors of the present invention have surprisingly found that the MFGM has a function to keep expression levels of genes encoding small proline-rich proteins (Sprr genes) and a gene encoding involucrin (Ivl gene) at normal levels. These genes are components of a cornified cell envelope in a horny layer located at the outermost of the skin tissue and are the most important for control of the TEWL. Until now, any finding that a specific component in milk has an effect on the small proline-rich proteins and involucrin has not been known.

The present invention has been completed based on such findings.

That is, a first aspect of the present invention provides a skin function-improving composition for oral ingestion, in which the composition contains a milk fat globule membrane (MFGM) as an active ingredient.

A second aspect of the present invention provides a skin function-improving composition according to the first aspect, in which the milk fat globule membrane (MFGM) is a product prepared by microfiltration.

A third aspect of the present invention provides a skin function-improving composition according to the first or second aspect, in which the milk fat globule membrane (MFGM) is a product prepared by centrifuging a retentate of microfiltration.

A fourth aspect of the present invention provides a skin function-improving composition according to any one of the first to third aspect, in which the milk fat globule membrane (MFGM) is a product obtained by a defatting treatment.

A fifth aspect of the present invention provides a skin function-improving composition according to any one of the first to fourth aspects, in which the milk fat globule membrane (MFGM) is a product obtained by a treatment with a protease.

A sixth aspect of the present invention provides a skin function-improving composition according to any one of the first to fifth aspects, in which the milk fat globule membrane (MFGM) is a product obtained from one or more selected from the group of milk, cream, buttermilk, whey, and processed products thereof.

A seventh aspect of the present invention provides a skin function-improving composition according to any one of the first to sixth aspects, in which the content of the milk fat globule membrane (MFGM) is 0.004% by mass or more.

An eighth aspect of the present invention provides a skin function-improving composition according to any one of the first to seventh aspects, in which the skin function-improving composition has an activity of improving a dry condition of the skin.

A ninth aspect of the present invention provides a skin function-improving composition according to any one of the first to eighth aspects, in which the skin function-improving composition has an activity of reducing a trans epidermal water loss.

A tenth aspect of the present invention provides a skin function-improving composition according to any one of the first to ninth aspects, in which the skin function-improving composition has an activity of suppressing epidermal thickness.

An eleventh aspect of the present invention provides a skin function-improving composition according to any one of the first to tenth aspects, in which the skin function-improving composition has an activity of normalizing expression levels of genes encoding small proline-rich proteins (Sprr genes) and a gene encoding involucrin (Ivl gene), which are components of a cornified cell envelope in a horny layer.

A twelfth aspect of the present invention provides a food or beverage, that contains the composition according to any one of the first to eleventh aspects, in which the food or beverage has a skin function-improving activity.

A thirteenth aspect of the present invention provides a skin function-improving agent for oral ingestion, that contains the composition according to any one of the first to eleventh aspects.

A fourteenth aspect of the present invention provides a skin function-improving agent for oral ingestion, that contains the milk fat globule membrane (MFGM) as an active ingredient.

### ADVANTAGEOUS EFFECTS OF INVENTION

According to the present invention, it is possible to provide the skin function-improving composition ‘which is inexpensive and highly safe and can be orally ingested’ and ‘which has an activity of improving a dry condition of the skin.’

Specifically, according to the present invention, it is possible to improve the dry condition of the skin by decreasing a TEWL of the skin or by suppressing skin thickness. In particular, in the case where maturation of a horny layer is inhibited by an abnormal increase in expression levels of genes encoding small proline-rich proteins (Sprr genes) and a gene encoding involucrin (Ivl gene), which are components of a cornified cell envelope in a horny layer in rough skin having an increased TEWL or skin thickness, oral administration of the MFGM can normalize the expression levels of the genes and stabilize the structure of the horny layer, resulting in improving the rough skin or a skin barrier function.

As a result, the present invention can provide the food or beverage and the skin function-improving agent for oral ingestion each having the skin function-improving activity.

### BRIEF DESCRIPTION OF DRAWING

FIG. 1 is a graph showing changes in expression levels of Sprr genes and Ivl gene by ingestion of the MFGM in Example 3.

### DESCRIPTION OF EMBODIMENTS

Hereinafter, the present invention is described in detail.

The present invention is a skin function-improving composition for oral ingestion, which contains the milk fat globule membrane (hereinafter, may be simply abbreviated as MFGM) as an active ingredient.

It should be noted that the milk fat globule membrane is usually abbreviated as MFGM in many cases, but in recent years, the membrane is also referred to as a milk lipid globule membrane and may be abbreviated as MLGM.

The MFGM that may be used for the present invention is not limited to one milked from a cow, and the MFGM separated from milk of, for example, a human, a goat, a horse, a camel, or a sheep may be used. In particular, one from milk of a cow is preferred.

The MFGM that may be used for the present invention may be obtained from one or more selected from the group of milk, cream, buttermilk, whey, and processed products thereof.

The MFGM exists in a small amount in each of, for example, milk, buttermilk, cream, whey, and processed products thereof, i.e., milk powder, concentrated buttermilk, buttermilk powder, whey powder, and concentrated whey.

In particular, the MFGM is preferably separated from buttermilk or a processed product thereof, i.e., concentrated buttermilk or buttermilk powder.

Therefore, in the case of using ‘milk’ as a raw material, the MFGM is preferably prepared in the following manner. That is, ‘cream’ is prepared with a cream separator, the prepared cream is churned to separate it into a butter fraction and a buttermilk fraction, and then the ‘buttermilk’ is used to prepare the MFGM.

Further, examples of buttermilk suitable for the present invention include buttermilk generated during the production of butter, or reconstituted buttermilk obtained by reconstituting buttermilk powder or concentrated buttermilk with water.

It has been known that the MFGM derived from milk can be prepared from the above-mentioned raw materials by a variety of methods, and specifically, the MFGM can be prepared by microfiltration.

The MFGM can be prepared by, for example, adjusting the above-mentioned raw material with hot water so as to have the total solid matter content of 1 to 15% (for example, about 6%), and holding at 20 to 65° C. (for example, about 50° C.) to dissolve the MFGM uniformly. In the case where buttermilk is used as the raw material, it is not necessary to adjust the solid matter content.

After that, the mixture is subjected to microfiltration using a microfiltration machine (MFS-1: manufactured by NIHON TETRA PAK) equipped with a microfiltration membrane of 0.5 to 6.0 μm (in particular, 0.8 μm), to thereby obtain a filtered retentate containing the MFGM at a high content.

Then, the resultant filtered retentate is dried (specifically, freeze-dried), to thereby obtain ‘MFGM powder.’ In particular, when the process is performed under conditions near the above-mentioned values within the parentheses, highly pure MFGM powder can be prepared.

It should be noted that more highly-pure MFGM powder can be obtained by: if necessary, centrifuging the resultant filtered retentate at 15,000×g for 15 minutes or more; and drying (specifically, freeze-drying) the resultant precipitate or redissolving the precipitate to an appropriate concentration and drying (specifically, freeze-drying or spray-drying) the mixture.

It should be noted that, if the MFGM obtained in the foregoing is subjected to a defatting treatment, it is possible to decrease the content of impurities derived from lipids and to improve the purity. The defatting treatment may be performed using an organic solvent such as chloroform, hexane, ethanol, or acetone. The treatment can be performed by: mixing the MFGM with such organic solvent; subjecting the mixture to operations such as upside-down mixing and centrifugation to thereby remove the solvent and obtain an aqueous layer.

Meanwhile, the MFGM obtained in the foregoing has insolubility in water. Therefore, it is preferable to improve the solubility in water by performing a treatment with a protease to decompose proteins into peptides in terms of, for example, processing into a food or beverage or a drug. The treatment with the protease may be performed so as to decompose the MFGM into a product having an average molecular weight of about 5,000 or less, preferably about 1,000 or less. Any protease may be used as long as the protease has such an activity that allows decomposition into a product in the above-mentioned range. For example, there may be used Sumizyme FP (manufactured by Shin Nihon Chemical Co., Ltd.), Sumizyme LP-50D (manufactured by Shin Nihon Chemical Co., Ltd.), Sumizyme FLAVOR (manufactured by Shin Nihon Chemical Co., Ltd.), Sumizyme TP (manufactured by Shin Nihon Chemical Co., Ltd.), Umamizyme G (manufactured by Amano Enzyme Inc.), and Protamex (manufactured by novozymes) alone or in combination of two or more thereof.

The MGFM obtained as described above has a ‘skin function-improving activity’ when orally ingested.

The skin function-improving activity in the present invention specifically includes an ‘activity of improving the dry condition of the skin’ and more specifically includes an ‘activity of reducing the TEWL of the skin’ and an ‘activity of suppressing skin thickness. Further, the activity provides an effect of improving the rough skin or skin-barrier function.

It should be noted that, in the case where maturation of the horny layer is inhibited by an abnormal increase in expression levels of genes encoding small proline-rich proteins (Sprr genes) and a gene encoding involucrin (Ivl gene), which are components of a cornified cell envelope in the horny layer, in the rough skin having increased TEWL, oral ingestion of the MFGM probably provides the above-mentioned activity of reducing the TEWL by normalizing the expression levels of the genes to stabilize the structure of the horny layer.

With regard to an effective dose of the skin function-improving composition containing the MFGM as an active ingredient of the present invention, the below-mentioned animal test using nude mice shows that, when the MFGM is orally ingested in an amount of 8.3 mg or more, preferably 25 mg or more per 1 kg of mouse body weight, the TEWL decreases.

Therefore, when the MFGM is usually ingested in an amount of 0.5 g or more, preferably 1.5 g or more per adult with a body weight of 60 kg per day, it is expected to improve the dry condition of the skin and to improve the rough skin or skin-barrier function. Therefore, the MFGM may be ingested so as to secure the required amount.

The MFGM may be contained in the skin function-improving composition so as to secure the above-mentioned dose, and the MFGM may be contained in an amount of specifically 0.004% by mass or more, preferably 0.01% by mass or more, more preferably 0.5% by mass or more, particularly preferably 1.5% by mass or more.

Meanwhile, there is no particular upper limit of the content, but the MFGM may be contained in an amount acceptable for the ratio to another raw material component depending on the embodiment. For example, the MFGM may be contained at a content of 70% by mass, specifically 32% by mass.

The skin function-improving composition containing the MFGM as an active ingredient of the present invention may be formed into a food, a pharmaceutical preparation, a cosmetic, an industrial raw material, or the like.

That is, the MFGM obtained as described above may be mixed with a variety of raw materials to produce a food, a pharmaceutical preparation, a cosmetic, an industrial raw material, or the like.

In the present invention, the shape of the food, the pharmaceutical preparation, the cosmetic, the industrial raw material, or the like is not particularly limited.

For example, the food may formed into a shape of a dairy product such as yogurt, milk beverage, or ice cream as well as another food that uses a dairy product as a raw material, such as cookie, spread, or bread, or a supplement as means for directly ingesting the MFGM.

Moreover, the food may be formed into a nutritional food by being mixed with, as other raw materials, raw materials generally used for other foods or beverages, such as sugars, lipids, proteins, vitamins, minerals, and flavors, and the like.

Further, the composition may be formed into a pharmaceutical preparation such as a powder, ground particles, a granule, a capsule in which the composition is filled, a solution where the composition is dispersed in water, a tablet obtained by blending the composition with a filler and the like.

### EXAMPLES

Hereinafter, the present invention is described by way of examples and the like, but the scope of the present invention is by no means limited thereto. (It should be noted that the following rates of the contents represented simply as “%” mean “% by mass”.)

### Example 1

### Effect of Decreasing TEWL by the MFGM Using Mice

The effect of decreasing the TEWL by the MFGM was examined as follows.

### (1) Preparation of the MFGM Powder

First, the MFGM powder was prepared by the following method. That is, ‘concentrated buttermilk’ (manufactured by Yotsuba Milk Products Co., Ltd., total solid matter content 30%) was diluted with hot water so that the total solid matter content was 6%, and holding at 50° C. After that, the mixture was subjected to microfiltration using a microfiltration machine (MFS-1: manufactured by NIHON TETRA PAK) equipped with a microfiltration membrane of 0.8 μm, and the filtered retentate was freeze-dried, to thereby prepare ‘MFGM powder.’

The ‘MFGM powder’ was found to contain proteins (86%), lipids (6%), carbohydrates (1%), and ash (7%) relative to the total solid matter, while the original proteins composing the MFGM have insolubility in water. Therefore, if the amount of soluble proteins (mainly whey proteins) in the powder is quantified, it is possible to estimate the purity of the original proteins composing the MFGM in the powder from the weight of protein components. In fact, the percentage of the soluble proteins in the ‘MFGM powder’ was 50%, and hence the original proteins composing the MFGM was found to have a purity of about 50%.

Meanwhile, as a control sample, a ‘milk protein’ product obtained by removing carbohydrates from skim milk (manufactured by Yotsuba Milk Products Co., Ltd.) was used. The milk protein product was found to contain proteins (86%), lipids (2%), carbohydrates (5%), and ash (7%) relative to the total solid matter.

### (2) Measurement of TEWL

The samples were used to compare the effect of decreasing the water loss by the MFGM with that of the milk protein product in the following TEWL measurement system.

Seventy-five 4-week-old Hos:HR-1 male mice (hereinafter, abbreviated as HR-1) were purchased from Hoshino Laboratory animals, inc. It has been known that, when the mice ingest a purified diet for HR-AD (manufactured by Nosan Corporation) as a specialty feed, the TEWL of the skin increases to develop atopic dermatitis-like.

During a pre-breeding and experiment period, polycarbonate cages were set on a stainless-steel rack in an environment of temperature 22±3° C., humidity 50±20%, ventilations 13 to 17 times/hour, and lighting 12 hours (8:00 to 20:00), and the mice were individually placed. During the pre-breeding and experiment period, the mice were allowed to freely ingest the diet using a stainless-steel feeder equipped with a polycarbonate dome-shaped feeder cover. As for water, the mice were allowed to drink tap water freely using a polysulfone waterer (with a stainless-steel tube). On the next day of arrival of the mice, the body weights of all the mice were measured. During a quarantine and habituation period, all the mice were allowed to ingest a normal feed.

Labo MR stock powder (manufactured by Nosan Corporation) was used as the normal feed and given to the mice in all the groups during the pre-breeding period, and after group division, given only to the mice in the non-treated group. Further, in order to increase the TEWL of each mouse, the purified diet for HR-AD (manufactured by Nosan Corporation) used as a specialty feed was given to all the mice excluding the non-treated group after group division as a mixed feed by blending with the respective test substance in a predetermined amount. The component list is as shown in Table 1 below.

TABLE 1

| | | Mixing ratio (%, wt/wt) | | | |
|---|---|---|---|---|---|
| | | | | | Milk |
| | | Normal | Specialty | 50% MFGM | protein |
| Group | Feeds | feed | feed | powder | product |
| Non-treated group | Labo MR stock | 100 | 0 | 0 | 0 |
| | powder | | | | |
| Control group | Purified diet | 0 | 97 | 0 | 3 |
| | for HR-AD | | | | |
| 1.5% | Purified diet | 0 | 97 | 3 | 0 |
| MFGM-administered | for HR-AD | | | | |
| group | (1.5% MFGM) | | | | |
| 0.0125% | Purified diet | 0 | 97 | 0.025 | 1.975 |
| MFGM-administered | for HR-AD | | | | |
| group | (0.0125% MFGM) | | | | |
| 0.00415% | Purified diet | 0 | 97 | 0.0083 | 1.9917 |
| MFGM-administered | for HR-AD | | | | |
| group | (0.00415% MFGM) | | | | |

At the last day of the quarantine and habituation period, the body weights of five-week-old HR-1 mice and trans epidermal water losses (hereinafter, may be abbreviated as TEWLs) of the dorsal skins of the mice were measured. The TEWLs were measured using Tewameter TM300 (manufactured by Courage+Khazaka). Further, the body weights were measured.

The mice which were evaluated to be healthy based on body weight increases and performance status observation during the quarantine and habituation period were divided into groups based on the grouping list in Table 1 by a stratified continuous randomization method using the TEWL values measured as indices.

On the next day of group division, ingestion of the specialty feed containing a predetermined amount of a substance to be administered was started in all the groups excluding the non-treated group, and the mice were allowed to ingest the feed freely until the day of euthanasia of the mice and removal of the skin. The mice in the non-treated group were continuously fed with the normal feed. The body weights, amounts of the feed ingested, and TEWLs of the dorsal skin were measured once a week after the start of ingestion of the specialty feed.

From the resultant values, mean values and standard errors were calculated for the respective groups.

The results were tested by the F-test to test equal variance, and significant differences between the non-treated group and the control group were determined by Student's t-test in the case of equal variance, or by Aspin-Welch's t-test in the case of unequal variance.

Significant differences between the control group and each of the skin function-improving composition groups were determined by the Bartlett method to test equal variance. In the case of equal variance, one-way analysis of variance was performed, and if there was a significant difference, mean values were compared by Dunnett's method. In the case of unequal variance, Kruskal-Wallis's H-test was performed, and if there was a significant difference, averages of the ranks were compared by Dunnett's method. In the cases of the F-test, Bartlett method, one-way analysis of variance, and Kruskal-Wallis's H-test, the level of significance was defined as P<0.05, while in the cases of the t-test and Dunnett's method, the levels of significance were defined as P<0.05 or P<0.01.

During the six-week breeding, there was confirmed no differences among the groups in the body weights and amounts of the diet ingested. The changes in the TEWL levels are shown in Table 2 below. It should be noted that each of the values represents a mean±standard error of 12 cases.

In each MFGM-administered group, the symbols ‘##’ and ‘#’ mean that there are significant differences in Dunnett's multiple comparison test at P<0.01 and at P<0.05, respectively, as compared with the control group (administered only the purified diet for HR-AD).

In the control group, the symbol ‘**’ means that there is a significant difference in the t-test at P<0.01 as compared with the non-treated group (administered only the normal feed).

TABLE 2

| | TEWL (g/m² · hr) | | | | | | |
|---|---|---|---|---|---|---|---|
| Group | 0 weeks | 1 week | 2 weeks | 3 weeks | 4 weeks | 5 weeks | 6 weeks |
| Non-treated group | 3.20 ± 0.12 | 3.33 ± 0.12 | 3.11 ± 0.12 | 3.66 ± 0.10 | 3.91 ± 0.10 | 3.57 ± 0.13 | 3.32 ± 0.13 |
| Control group | 3.22 ± 0.12 | 3.14 ± 0.15 | 3.39** ± 0.11 | 4.35** ± 0.14 | 5.82** ± 0.42 | 7.07** ± 0.29 | 7.22** ± 0.22 |
| 1.5% | 3.22 ± 0.13 | 3.51 ± 0.22 | 3.37 ± 0.12 | 4.26 ± 0.29 | 3.81## ± 0.23 | 4.54## ± 0.18 | 4.52## ± 0.24 |
| MFGM-administered group | | | | | | | |
| 0.0125% | 3.21 ± 0.12 | 3.09 ± 0.11 | 3.53 ± 0.15 | 4.00 ± 0.21 | 4.93 ± 0.23 | 6.00 # ± 0.18 | 6.18## ± 0.20 |
| MFGM-administered group | | | | | | | |
| 0.00415% | 3.21 ± 0.12 | 3.30 ± 0.17 | 3.57 ± 0.11 | 4.37 ± 0.24 | 5.78 ± 0.42 | 6.73 ± 0.31 | 6.29 # ± 0.25 |
| MFGM-administered group | | | | | | | |

The results show that, in the case of the ‘control group’ (administered only the purified diet for HR-AD), the TEWLs increased markedly from after the lapse of two weeks to after the lapse of six weeks, and at all the points after the lapse of two weeks, the values were significantly high compared with the non-treated group. This shows that the moisturizing ability of the dorsal skin of the HR-1 mice was induced to be lowered by ingestion of the specialty feed.

On the other hand, the TEWLs of the ‘1.5% MFGM-administered group’ were significantly lower than those of the control group after the lapse of four, five, and six weeks; the TEWLs of the ‘0.0125% MFGM-administered group’ were significantly lower than those of the control group after the lapse of five and six weeks; and the TEWLs of the ‘0.00415% MFGM-administered group’ were significantly lower than those of the control group after the lapse of six weeks.

The TEWLs of the groups in which the MFGM was ingested were found to depend on the doses. The results show that the MFGM suppresses an increase in the TEWL of the dorsal skin of the HR-1 mice and improves impairment of the moisturizing ability.

### Example 2

### Effect of Suppressing Skin Thickness by the MFGM Using Mice

Example 1 has shown that the TEWL is decreased by the MFGM. In view of this, the dorsal skin was removed after the lapse of six weeks. A part of the resultant dorsal skin was fixed with 10% neutral buffered formalin in accordance with a conventional method, and hematoxylin-eosin staining was performed, followed by a histopathological examination. Based on the histopathological image obtained by the histopathological examination, the thicknesses of epidermis and dermis in the each mouse's dorsal skin were measured. In addition, the thickness of skin was defined as a sum of the thickness of epidermis and the thickness of dermis.

The thicknesses of epidermis, dermis, and skin in the dorsal are shown in Table 3 below. It should be noted that each of the values represents a mean±standard error of 12 cases.

Further, in each MFGM-administered group, the symbol ‘##’ means that there is a significant difference in Dunnett's multiple comparison test at P<0.01 as compared with the control group (administered only the purified diet for HR-AD). It should be noted that, in the control group, the symbol ‘**’ means that there is a significant difference in the t-test at P<0.01 as compared with the non-treated group (administered only the normal feed).

TABLE 3

| | Thickness of the dorsal skin | | |
|---|---|---|---|
| | after lapse of six weeks | | |
| | Thickness of | Thickness of | Thickness of |
| Group | epidermis (μm) | dermis (μm) | skin (mm) |
| Non-treated group | 13.8 ± 0.7 | 290.4 ± 16.3 | 0.30 ± 0.02 |
| Control group | 56.3** ± 2.8 | 379.6** ± 14.8 | 0.44** ± 0.02 |
| 1.5% | 25.0## ± 2.3 | 292.9## ± 12.5 | 0.32## ± 0.01 |
| MFGM-administered | | | |
| group | | | |
| 0.0125% | 41.7## ± 2.1 | 318.3## ± 13.0 | 0.36## ± 0.01 |
| MFGM-administered | | | |
| group | | | |
| 0.00415% | 46.7## ± 2.2 | 332.1## ± 10.9 | 0.38## ± 0.01 |
| MFGM-administered | | | |
| group | | | |

The results show that in the ‘control group’ (administered only the purified diet for HR-AD), the thickness of epidermis was 56.3±2.8 μm, the thickness of dermis was 379.6±14.8 μm, and the thickness of skin was 0.44±0.02 mm, and the thicknesses of epidermis, dermis, and skin were each significantly larger than that of the non-treated group. Therefore, it was found that skin thickness was induced in the control group.

On the other hand, in the ‘1.5% MFGM-administered group,’ the thickness of epidermis was 25.0±2.3 μm, the thickness of dermis was 292.9±12.5 μm, and the thickness of skin was 0.32±0.01 mm.

Further, in the ‘0.0125% MFGM-administered group’, the thicknesses of epidermis, dermis, and skin were respectively 41.7±2.1 μm, 318.3±13.0 μm, and 0.36±0.01 mm, while in the ‘0.00415% MFGM-administered group’, the thicknesses of epidermis, dermis, and skin were respectively 46.7±2.2 μm, 332.1±10.9 μm, and 0.38±0.01 mm. That is, in all the MFGM-administered groups, the thicknesses of epidermis, dermis, and skin were each significantly smaller than that of the control group and became smaller depending on the doses.

The results show that the MFGM suppresses the skin thickness in the dorsal of the HR-1 mouse and improved the skin to be a normal skin.

### Example 3

### Analysis of Gene of Mouse Having Decreased TEWL and Suppressed Skin Thickness by the MFGM

Example 1 and Example 2 have revealed that the MFGM decreases the TEWL and suppresses the skin thickness. In this case, expression levels of the Ivl gene and Sprr genes expressed in the skin of mice were analyzed by real-time PCR.

First, mice were bred with administration in almost the same way as in the test in Example 1. It should be noted that the mice were divided into three groups each including 6 mice, i.e., ‘non-treated group,’ ‘control group,’ and ‘1.5% MFGM-administered group.’

Then, after a lapse of 4 weeks, RNA was extracted from the dorsal skin of each mouse in accordance with a conventional method. The value of the TEL after the lapse of 4 weeks was almost the same as that in Table 2. The resultant RNA was subjected to a reverse transcription reaction with a PrimeScript® RT reagent Kit (Perfect Real Time) (manufactured by TAKARA BIO INC.) to provide cDNA. Real-time PCR was performed targeting involucrin gene (“Ivl”) and small proline-rich protein genes (“Sprr1b,” “Sprr2a,” “Sprr2d,” “Sprr2e,” “Sprr2f,” “Sprr2g,” and “Sprr2h”), all of which are components of the cornified cell envelope in a horny layer.

For the real-time PCR reaction, an SYBR® Premix EX TaqTMII (Perfect Real Time) (manufactured by TAKARA BIO INC.) was used, and for the PCR device and detector, a DNA Engine Opticon™ System <PTC-200 DNA Engine™ Cycler, CFD-3200 Opticon™ Detector> (manufactured by MJ Research, Inc.) was used.

As the expression level of each gene, an amount relative to the glucuronidase-β gene (Gusb, internal standard gene) was determined and compared as a relative value based on the expression level of the control group, defined as 1.

Table 4 shows a relationship between genes used in the expression analyses and primer sequences used (sequence numbers in the following sequence listing).

The results were tested by the F-test to test equal variance, and significant differences between the groups were determined by Student's t-test in the case of equal variance, or by Welch's t-test in the case of unequal variance.

TABLE 4

| Target | | | | |
|---|---|---|---|---|
| gene | Primer set | | | |
| name | Forward primer | | Reverse primer | |
| Ivl | Ivl-F | SEQ ID NO: 1 | Ivl-R | SEQ ID NO: 2 |
| Sprr1b | Sprr1b-F | SEQ ID NO: 3 | Sprr1b-R | SEQ ID NO: 4 |
| Sprr2a | Sprr2a-F | SEQ ID NO: 5 | Sprr2a-R | SEQ ID NO: 6 |
| Sprr2d | Sprr2d-F | SEQ ID NO: 7 | Sprr2d-R | SEQ ID NO: 8 |
| Sprr2e | Sprr2e-F | SEQ ID NO: 9 | Sprr2e-R | SEQ ID NO: 10 |
| Sprr2f | Sprr2f-F | SEQ ID NO: 11 | Sprr2f-R | SEQ ID NO: 12 |
| Sprr2g | Sprr2g-F | SEQ ID NO: 13 | Sprr2g-R | SEQ ID NO: 14 |
| Sprr2h | Sprr2h-F | SEQ ID NO: 15 | Sprr2h-R | SEQ ID NO: 16 |
| Gusb | Gusb-F | SEQ ID NO: 17 | Gusb-R | SEQ ID NO: 18 |

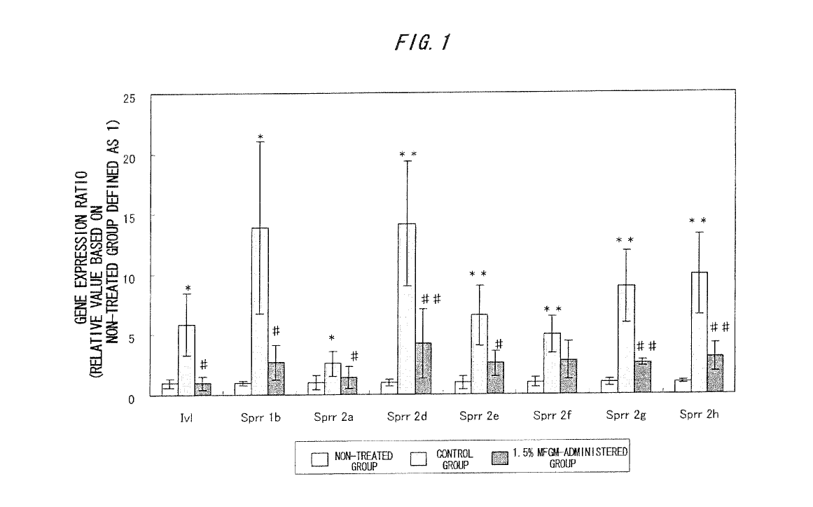

The resultant expression levels are shown in Table 5 and FIG. 1. It should be noted that, in comparisons of the gene expression levels in Table 5 and FIG. 1, the symbols ‘**’ and ‘*’ mean that there are significant differences in the t-test at P<0.01 and at P<0.05, respectively, when the control group is compared with the non-treated group, while the symbols ‘##’ and ‘#’ mean that there are significant differences in the t-test at P<0.01 and at P<0.05, respectively, when the 1.5% MFGM-administered group is compared with the control group (administered only the purified diet for HR-AD).

TABLE 5

| | Gene expression ratio (relative value) | | | | |
|---|---|---|---|---|---|
| Group | IVl | Sprr1b | Sprr2a | Sprr2d | Sprr2e |
| Non-treated | 1.000 ± 0.385 | 1.000 ± 0.156 | 1.000 ± 0.589 | 1.000 ± 0.252 | 1.000 ± 0.522 |
| group | | | | | |
| Control group | 5.883* ± 2.561 | 13.883* ± 7.153 | 2.595* ± 1.037 | 14.147** ± 5.155 | 6.539** ± 2.476 |
| 1.5% MFGM- | 0.988# ± 0.578 | 2.670# ± 1.427 | 1.447# ± 0.926 | 4.213## ± 2.897 | 2.571# ± 1.036 |
| administered | | | | | |
| group | | | | | |

| | Gene expression ratio (relative value) | | |
|---|---|---|---|
| Group | Sprr2f | Sprr2g | Sprr2h |
| Non-treated | 1.000 ± 0.404 | 1.000 ± 0.259 | 1.000 ± 0.136 |
| group | | | |
| Control group | 4.961** ± 1.515 | 8.893** ± 2.964 | 9.869** ± 3.306 |
| 1.5% MFGM- | 2.810 ± 1.578 | 2.613## ± 0.237 | 3.065## ± 1.176 |
| administered | | | |
| group | | | |

As shown in Table 5 and FIG. 1, there were confirmed significant differences in the expression levels of the Ivl gene and Sprr genes.

In the case of the ‘control group’ (group in which the purified diet for HR-AD developing atopic dermatitis-like was ingested and TEWL increased), the expression levels of all the genes were significantly enhanced compared with the ‘non-treated group’ in which the normal diet was ingested (group which ingested the usual diet).

On the other hand, in the case of the ‘1.5% MGFM-administered group’ (group in which the purified diet for HR-AD supplemented with 1.5% MFGM was ingested and the TEWL was almost same as the non-treated group), the expression levels of the Ivl gene and all the Sprr genes excluding Sprr2f were significantly low compared with the ‘control group’ and approached the expression levels of the ‘non-treated group.’ Moreover, although there was no significant difference, Sprr2f exhibited a similar behavior to those of the other genes.

This shows that the MFGM orally ingested has an activity of returning the expression levels of the overexpressed Ivl gene and Sprr genes to normal levels in the skin having increased TEWL due to a loss of skin homeostasis by dermatitis.

The results suggest that the increase in the TEWL was suppressed by returning the structure of the horny layer to a normal state to restore the function of the horny layer in the skin where maturation of the horny layer was inhibited.

The MFGM has not heretofore been known to have such functions on the Ivl gene and Sprr genes.

### Example 4

### Preparation of Yogurt Containing the MFGM

A yogurt containing the MFGM was prepared in the following manner.

Specifically, the MFGM powder was suspended in water so as to have a ratio of 2.5% (w/w) and, after being heated to 40° C., dissolved with a cutter mixer for 10 minutes at 10,000 rpm. Raw materials were blended (final concentration of MFGM: 0.5%) based on each of the recipe of ‘low-fat yogurt’ (Table 6) and ‘fat-free yogurt’ (Table 7) shown below, and heated to 60° C.

The mixture was homogenized with a homogenizer at 150 kg/cm², then subjected to a pasteurization step at 90° C. for 10 minutes, and cooled to 43° C.

A starter (Yo-MIX 499) was inoculated at 0.15 DCU/kg, filling was performed so as to achieve 100 g/container, and culture was performed at 43° C. When the pH reached 4.7 or less, the container was cooled to 10° C. or less and refrigerated.

TABLE 6
Low-fat yogurt

| Raw material name | Composition % |
|---|---|
| 47% Fresh cream | 2.5 |
| Skim milk | 9.0 |
| 2.5% MFGM solution | 20.0 (Final concentration 0.5%) |
| Beet granulated sugar | 6.0 |
| Milk flavor | 0.1 |
| Water | 62.4 |
| Total | 100.0 |
| (Starter) | (0.15 DCU/kg) |

TABLE 7
Fat-free yogurt

| Raw material name | Composition % |
|---|---|
| 47% Fresh cream | 0.4 |
| Skim milk | 9.8 |
| 2.5% MFGM solution | 20.0 (Final concentration 0.5%) |
| Beet granulated sugar | 6.0 |
| Milk flavor | 0.1 |
| Water | 63.7 |
| Total | 100.0 |
| (Starter) | (0.15 DCU/kg) |

As a result, also in the case of each of the low-fat and fat-free yogurts, a trial product having a very good flavor was obtained.

The yogurts prepared as described above are expected to have effects of reducing the TEWL to keep moisturizing the skin, improving the dry condition of the skin, and improving the rough skin or skin-barrier function.

### Example 5

### Preparation of Supplement Containing the MFGM

A supplement containing the MFGM was prepared as follows.

That is, based on the following composition list (Table 8), raw materials were mixed, and the tablets were prepared with a rotary tableting machine.

TABLE 8
Supplement

| Raw material name | Composition % |
|---|---|
| MFGM powder with purity of 50% | 62.5 (Final concentration 31.25%) |
| Milk calcium | 18.0 |
| Reconstituted maltose starch syrup | 18.0 |
| Sucrose fatty acid ester | 1.5 |
| Total | 100.0 |

The supplement prepared as described above is expected to have effects of reducing the TEWL to keep moisturizing the skin, improving the dry condition of the skin, and improving the rough skin or skin-barrier function.

### Example 6

### Effect of Improving Human Skin by the MFGM

### (1) Ingestion Test of MFGM-Containing Food

Forty 20- to 40-year-old healthy males and females, who were always conscious of their skin dryness and had no problems in skin measurement, physical examination, evaluation by a doctor, and blood and urine analysis, were selected as test subjects. It should be noted that this test was performed after approval by the institutional review board in keeping with the ethical principles based on the Declaration of Helsinki and a clinical protocol in the gain of agreement of the test subjects and the overall test.

As for test foods, the MFGM supplement prepared based on the composition described in Example 5 was used as a subject food, and a product obtained by replacing the 50% MFGM powder by skim milk was used as a control food.

The test was performed by a double-blind method. First, the forty test subjects were divided into two groups. It should be noted that, at the first day of the start of the test (week 0), there were confirmed no significant differences between the groups in all the measurement items described below.

The test subjects were allowed to ingest the test food on consecutive 28 days (from Feb. 4, 2009 to Mar. 4, 2009 or from Feb. 5, 2009 to Mar. 5, 2009) at doses of five tablets in the morning, five tablets at noon, and six tablets at night, and examination and measurement were performed at the first day of ingestion (week 0), week 2, and week 4. Examination items are as shown below.

A) Evaluation by Doctor (at Time of Screening, Weeks 0, 2, and 4)

A medical interview and auscultation/percussion were performed by a doctor.

B) Physical Examination (at Time of Screening, Week 4)

A body height (only at time of screening), a body weight, a body fat percentage, a BMI, a body temperature, a blood pressure, and a beat were measured.

C) Hematological Examination (at Time of Screening, Weeks 0, 2, and 4)

A white blood cell count, a red blood cell count, hemoglobin, hematocrit, and a platelet count were measured using an automated hematology analyzer (SE9000) and an automated hematology analyzer (XE2100: manufactured by Sysmex Corporation).

D) Blood Biochemical Examination (at Time of Screening, Weeks 0, 2, and 4)

Total protein, A/G, albumin, total bilirubin, AST, ALT, LDH, γ-GTP, ALP, urea nitrogen, uric acid, creatinine, Na, K, Cl, fasting blood glucose, total cholesterol, HDL-cholesterol, and neutral fat were measured.

Only the fasting blood glucose value was measured using an automated analyzer JCA-BM9030 (manufactured by JEOL Ltd.). The other items were measured using an automated analyzer JCA-BM8060 (manufactured by JEOL Ltd.).

E) Urine Examination (at Time of Screening, Weeks 0, 2, and 4)

Sugar, protein, occult blood, urobilinogen, a pH, and a specific gravity were measured. A fully automated urine analyzer AUTION MAX AX-4280 (manufactured by ARKRAY, Inc.) was used for the measurement.

F) Measurement of Change in TEWL of Face (at the Time of Screening, Weeks 0, 2, and 4)

TEWLs were measured using Tewameter TM300 (manufactured by Courage+Khazaka), and habituation was performed in a constant temperature and humidity room of 24±2° C. and 55±5% for 20 minutes or more. The measurement site was 1 cm below the left eye.

In addition, the TEWL before ingestion of the test food was defined as 0, and time-dependent changes were determined as changes in TEWLs.

G) Questionnaire Survey (Weeks 0, 2, 3, and 4)

A questionnaire survey was performed on the dryness, skin roughness, vitality, moisture feeling, complexion, wrinkles, transparency, makeup easiness (only females) of the face skin.

Sensory levels were evaluated once every week based on the face skin condition using the following criteria; when the face skin condition before ingestion was defined as 0, the case where the condition was improved was evaluated as 2, the case where the condition was somewhat improved was evaluated as 1, the case of no change was evaluated as 0, the case where the condition was somewhat worsened was evaluated as −1, and the case where the condition was worsened was evaluated as −2.

H) Ingestion Record

Use of the test food, healthy food, and drugs and amount of alcohol intake (type and amount of alcohol) were recorded every day during the test period.

As criteria for exclusion of subjects after the start of the test, a person who ingested the test food at an ingestion rate of less than 90%, a person who ingested a contraindicated food or pharmaceutical preparation, and a person who performed crapulence which was clearly beyond a usual diet one day before the measurement were removed. As a result, it was found that a male and a female of the control food group and a female of the subject food group ingested a contraindicated pharmaceutical preparation. Therefore, in this test, all the data of the three persons were removed before the analysis.

The ingestion rate of the control food group was 100%, while the ingestion rate of the subject food group was 99.4% (maximum 100%, minimum 92.9%).

In addition, the resultant values for the respective examination items are represented as a mean±standard deviation for each group. In order to compare the test foods for each item, a test of equal variance was performed by the F-test, and in the case of equal variance, evaluation was performed by Student's t-test, while in the case of unequal variance, evaluation was performed by Aspin-Welch's t-test. In order to compare a value before ingestion with a value at each ingestion week in each group, evaluation was performed by the paired t-test. A significance level of the F-test was 5% on the both sides, while significance levels of the t-test were 5% and 1% on the both sides.

### (2) Results

As a result of the test, there were confirmed significant differences between the both groups (the subject food group and the control food group) in the changes in the TEWLs and the results of the questionnaire survey on the skin.

It should be noted that, from the results of the physical examination, hematological examination, blood biochemical examination, urine examination, and findings of evaluation by a doctor, there was confirmed no adverse event derived from the MFGM.

First, the results of the changes in the TEWLs are shown in Table 9.

It should be noted that, a larger change means that the skin was changed into one where water is more easily lost, while a smaller change means that the ability to retain water is stable or improved keep moisturizing the skin.

As compared with the value before ingestion, the changes in the water losses significantly increased in both the groups two weeks after ingestion. Four weeks after ingestion, the change in the water loss significantly increased in the control food group. This was probably caused by a seasonal factor to increase the TEWL because the test was performed at midwinter. However, four weeks after ingestion, the change in the water loss of the subject food group was significantly lower than that of the control food group. That is, in the case of the control food containing skim milk, the TEWL continued to increase due to the seasonal factor, while in the case of the subject food containing the MFGM, the TEWL started to decrease four weeks after ingestion. The fact shows that the MFGM has an activity of decreasing the TEWL also in human.

The results show that, when the subject food containing the MFGM is ingested for 4 weeks, an increase in the water loss can be suppressed, and particularly show that, in winter when the skin tends to dry and causes trouble of skin roughness, the skin can be maintained to be normal.

TABLE 9

| | Change in TEWL (g/hm²) | | |
|---|---|---|---|
| Group | Before ingestion | 2 weeks later | 4 weeks later |
| Control food | 0.00 ± 0.00 | 3.40 ± 3.29** | 4.21 ± 3.11** |
| Subject food | 0.00 ± 0.00 | 5.31 ± 4.22** | 1.53 ± 3.87# |

Control food = 18 subjects, subject food = 19 subjects
*,**showing a significant difference as compared with the value before ingestion (p < 0.05 and 0.01, respectively) (Paired-t)
#showing a significant difference as compared with control food (p < 0.05) (t-test)

Next, the results of the questionnaire survey on the skin are shown in Table 10.

As compared with the ‘Before ingestion,’ one week after ingestion, significant increase was observed with respect to the face dryness of the control food group and with respect to all the items other than the face transparency of the subject food group; two weeks after ingestion, significant increase was observed with respect to the face dryness and face roughness of the control food group and with respect to the face dryness, face roughness, and makeup easiness of the subject food group; three weeks after ingestion, significant increase was observed with respect to the face dryness of the control food group and with respect to all the items other than the face transparency of the subject food group; and four weeks after ingestion, significant increase was observed with respect to the face dryness of the control food group and with respect to all the items of the subject food group.

In addition, as compared with the ‘Control food group,’ significantly high values were observed in the subject food group with respect to the face roughness, face complexion, and makeup easiness three weeks after ingestion; and with respect to all the items four weeks after ingestion.

The results show that it is possible to actually feel an effect of improving impaired skin quality due to skin dryness by ingestion of the MFGM.

TABLE 10

| | | Before | 1 week | 2 weeks | 3 weeks | 4 weeks |
|---|---|---|---|---|---|---|
| Item | Group | ingestion | later | later | later | later |
| Face dryness | Control | 0.0 ± 0.0 | 0.4 ± 0.6** | 0.7 ± 0.8** | 0.4 ± 0.9* | 0.5 ± 0.9* |
| | food | | | | | |
| | Subject | 0.0 ± 0.0 | 0.6 ± 0.7** | 0.8 ± 0.5** | 0.8 ± 0.5** | 0.8 ± 0.8**# |
| | food | | | | | |
| Face | Control | 0.0 ± 0.0 | 0.1 ± 0.5 | 0.4 ± 0.6** | 0.2 ± 0.8 | 0.1 ± 0.8 |
| roughness | food | | | | | |
| | Subject | 0.0 ± 0.0 | 0.4 ± 0.6** | 0.4 ± 0.6* | 0.7 ± 0.7**# | 0.5 ± 0.9**# |
| | food | | | | | |
| Face | Control | 0.0 ± 0.0 | 0.2 ± 0.4 | 0.2 ± 0.5 | 0.1 ± 0.4 | 0.1 ± 0.5 |
| complexion | food | | | | | |
| | Subject | 0.0 ± 0.0 | 0.3 ± 0.6* | 0.3 ± 0.6 | 0.5 ± 0.6**# | 0.4 ± 0.7**## |
| | food | | | | | |
| Face | Control | 0.0 ± 0.0 | 0.0 ± 0.0 | 0.1 ± 0.3 | 0.1 ± 0.4 | 0.2 ± 0.5 |
| transparency | food | | | | | |
| | Subject | 0.0 ± 0.0 | 0.2 ± 0.5 | 0.2 ± 0.5 | 0.3 ± 0.7 | 0.4 ± 0.7**# |
| | food | | | | | |
| Makeup | Control | 0.0 ± 0.0 | 0.3 ± 0.6 | 0.3 ± 0.8 | 0.0 ± 0.8 | 0.3 ± 0.9 |
| easiness | food | | | | | |
| (only | Subject | 0.0 ± 0.0 | 0.6 ± 0.5** | 0.5 ± 0.5** | 0.6 ± 0.5**# | 1.0 ± 0.7**# |
| females) | food | | | | | |

Control food = 18 subjects (13 females), subject food = 19 subjects (12 females)
Mean ± standard deviation
Calculation was performed based on the following criteria with the value before ingestion defined as “0”: “better: 2,” “slightly better: 1,” “no change: 0,” “slightly worse: −1,” and “worse: −2.”
*,**showing a significant difference as compared with the value before ingestion (p < 0.05 and 0.01, respectively) (Paired-t)
#,##: showing a significant difference as compared with control food (p < 0.05 and 0.01, respectively) (t-test)

### Example 7

### Effect of the MFGM on Human Keratinocyte

### (1) Preparation of Enzymatically Decomposed and Defatted MFGM

4.23 g of MFGM powder prepared in the same manner as in Example 1 were added to 160 ml of water and sufficiently suspended using a Polytron homogenizer (19,500 rpm, 5 minutes). Then, 400 ml of methanol and 200 ml of chloroform were added and further suspended using a Polytron homogenizer (19,500 rpm, 10 minutes).

Subsequently, the suspension was transferred to a separating funnel, and 160 ml of water and 200 ml of chloroform were added thereto. The mixture was sufficiently blended by upside-down mixing and left to stand all night and all day. The organic solvent layer was removed, and 200 ml of chloroform were added to the aqueous layer. The mixture was sufficiently blended by upside-down mixing and left to stand all night and all day. The resultant aqueous layer was freeze-dried, to thereby obtain a MFGM protein fraction (defatted MFGM powder).

The resultant defatted MFGM powder has insolubility in water, and hence cannot be used in the following test unless proteins are decomposed into peptides with a protease to improve the solubility. Therefore, 3 g of the defatted MFGM powder were added to 30 ml of water and sufficiently suspended using a Polytron homogenizer (19,500 rpm, 5 minutes). 1% (v/v) Sumizyme FLAVOR (manufactured by Shin Nihon Chemical Co., Ltd.), 1% (w/w) Sumizyme TP (manufactured by Shin Nihon Chemical Co., Ltd.), and 1% (w/w) Protamex (manufactured by Novozymes) were added to the suspension, and the mixture was allowed to react at 55° C. for 1 hour. After completion of the reaction, the mixture was incubated in boiling water for 5 minutes to deactivate the enzymes. It should be noted that the same procedure was performed in parallel without addition of the defatted MFGM powder to prepare a product as a control.

It should be noted that when ‘enzymatically decomposed and defatted MFGM’ was subjected to peptide PAGE, the proteins contained were found to have an average molecular weight of about 1,000.

### (2) Culture of Human Epidermal Keratinocyte (HEK)

Human epidermal keratinocytes (HEKs) collected from the face of a 48-year-old Caucasian female and having the same lot were purchased from CELL APLLICATIONS, INC. The freeze-preserved HEKs were treated in accordance with a conventional method and inoculated into 35 mm dishes at 5,000 cells/cm² using a growth medium containing 0.05 mM calcium (containing Ca at a low content). It should be noted that, KERATINOCYTE CALCIUM-FREE MEDIUM (manufactured by CELL APLLICATIONS, INC.) was used as the growth medium, and a sterilized calcium chloride solution was used as a source of calcium in the medium.

The keratinocytes were divided into two groups including: a group of keratinocytes cultured with the addition of the above-mentioned ‘enzymatically decomposed and defatted MFGM’ in the growth medium at a final concentration of 100 μM (MFGM group); and a group of keratinocytes cultured with the addition of an enzymatically treated solution containing no MFGM (the above-mentioned control) in an equal amount (control group).

Both the groups were cultured by leaving the keratinocytes to stand still in a 37° C., 5% CO₂ incubator in a wet condition, and the medium was exchanged on alternate days.

When the cells reached about 60% confluence, the medium was exchanged for a differentiation-inducing medium (the above-mentioned growth medium containing 2 mM calcium). After a lapse of 0, 6, and 24 hours, the cells were collected and subjected to the following tests.

### (3) Confirmation of Gene Expression Levels by Real-Time PCR

RNA was extracted from HEK at various time periods after the exchange for the differentiation-inducing medium in accordance with a conventional method, and in the same manner as in Example 3, real-time PCR was performed targeting involucrin gene (“Ivl”) and small proline-rich protein genes (“Sprr1b,” “Sprr2a,” “Sprr2d,” “Sprr2e,” “Sprr2f,” “Sprr2g,” and “Sprr2h”), all of which are components of the cornified cell envelope in a horny layer.

As the expression level of each gene, an amount relative to a glyceraldehyde 3-phosphate dehydrogenase gene (Gapdh, internal standard gene) was determined and compared as a relative value based on the expression level of the control group, defined as 1.

Table 11 shows a relationship between genes used in the expression analyses and primer sequences used (sequence numbers in the following sequence listing).

The results were tested by the F-test to test equal variance, and significant differences between the groups were determined by Student's t-test in the case of equal variance, or by Welch's t-test in the case of unequal variance.

TABLE 11

| Target | | | | |
|---|---|---|---|---|
| gene | Primer set | | | |
| name | Forward primer | | Reverse primer | |
| Ivl | Ivl-F2 | SEQ ID NO: 19 | Ivl-R2 | SEQ ID NO: 20 |
| Sprr1a | Sprr1a-F2 | SEQ ID NO: 21 | Sprr1a-R2 | SEQ ID NO: 22 |
| Sprr1b | Sprr1b-F2 | SEQ ID NO: 23 | Sprr1b-R2 | SEQ ID NO: 24 |
| Sprr2a | Sprr2a-F2 | SEQ ID NO: 25 | Sprr2a-R2 | SEQ ID NO: 26 |
| Sprr2b | Sprr2b-F2 | SEQ ID NO: 27 | Sprr2b-R2 | SEQ ID NO: 28 |
| Sprr2c | Sprr2c-F2 | SEQ ID NO: 29 | Sprr2c-R2 | SEQ ID NO: 30 |
| Sprr2d | Sprr2d-F2 | SEQ ID NO: 31 | Sprr2d-R2 | SEQ ID NO: 32 |
| Sprr2e | Sprr2e-F2 | SEQ ID NO: 33 | Sprr2e-R2 | SEQ ID NO: 34 |
| Sprr2f | Sprr2f-F2 | SEQ ID NO: 35 | Sprr2f-R2 | SEQ ID NO: 36 |
| Sprr2g | Sprr2g-F2 | SEQ ID NO: 37 | Sprr2g-R2 | SEQ ID NO: 38 |
| Sprr3 | Sprr3-F2 | SEQ ID NO: 39 | Sprr3-R2 | SEQ ID NO: 40 |
| Sprr4 | Sprr4-F2 | SEQ ID NO: 41 | Sprr4-R2 | SEQ ID NO: 42 |
| Gapdh | Gapdh-F2 | SEQ ID NO: 43 | Gapdh-R2 | SEQ ID NO: 44 |

Table 12 shows the resultant expression levels. There were confirmed significant differences in Sprr3 and Sprr4, and hence the results of the two genes are shown. It should be noted that, in the comparison of the gene expression levels, the symbol ‘*’ means that there is a significant difference with respect to the control group at P<0.05 in the t-test.

TABLE 12

| | Gene expression ratio (relative value) | | | | | |
|---|---|---|---|---|---|---|
| | Sprr3 | | | Sprr4 | | |
| Group | 0 hours | 6 hours | 24 hours | 0 hours | 6 hours | 24 hours |
| Control | 1.000 ± 0.850 | 3.607 ± 1.956 | 3.785 ± 1.134 | 1.000 ± 0.422 | 2.763 ± 0.782 | 3.627 ± 0.838 |
| group | | | | | | |
| MFGM group | 0.487 ± 0.172 | 2,041 ± 0.481 | 1.410 ± 0.802* | 0.549 ± 0.174 | 2.799 ± 1.012 | 1.750 ± 0.800* |

The results show that, at the time immediately after exchange for the differentiation-inducing medium (0 hours), no significant difference was confirmed in the expression levels of both the genes Sprr3 and Sprr4 between the MFGM group and the control group, but the expression levels of the MFGM group were about half those of the control group.

In addition, after exchange for the differentiation-inducing medium, the expression levels of both the genes Sprr3 and Sprr4 of the control group were found to continue to increase, while in the case of the MFGM group, the expression levels increased once after a lapse of 6 hours but decreased to levels significantly lower than those of the control group after a lapse of 24 hours. Specifically, the Sprr3 expression level increased to a level about 0.37 time that of the control group, while the Sprr4 expression level increased to a level about 0.48 time that of the control group.

The above-mentioned results reveal that a peptide derived from the defatted MFGM has an activity of suppressing expression levels of the Sprr genes in the HEKs. Such findings have not heretofore been known.

### Example 8

### Preparation of Highly-Pure MFGM

The MFGM was prepared by the following method. That is, ‘concentrated buttermilk’ (manufactured by Yotsuba Milk Products Co., Ltd., total solid matter content 30%) was diluted with hot water so that the total solid matter content as 6%, and held at 50° C. After that, the mixture was subjected to microfiltration using a microfiltration machine (MFS-1: manufactured by NIHON TETRA PAK) equipped with a microfiltration membrane of 0.8 μm, and the filtered retentate was centrifuged using a cylindrical continuous centrifuge machine ASM100 (manufactured by TOMOE Engineering Co., Ltd.), to thereby obtain a precipitate.

The resultant precipitate was re-suspended so that the solid matter content was 2%, and freeze-dried, to thereby prepare highly pure ‘MFGM powder.’

### INDUSTRIAL APPLICABILITY

The present invention provides the composition for oral ingestion, which is a safe material which has been usually eaten, is more inexpensive, and has a skin function-improving effect.

Therefore, the skin function-improving composition according to the present invention is expected to have an effect of improving rough skin and skin barrier function by improving the dry condition of the skin when ingested as an everyday diet or the like.

In addition, the present invention is expected to provide a skin function-improving agent to provide the above-mentioned effects and to be applied to pharmaceutical fields.

1-14. (canceled)

15. A method for treating dry skin comprising administering to a patient in need thereof a pharmaceutically effective amount of a milk fat globule membrane.

16. The method according to claim 15, wherein the milk fat globule membrane is a product prepared by microfiltration.

17. The method according to claim 15, wherein the milk fat globule membrane is a product prepared by centrifuging a retentate of microfiltration.

18. The method according to claim 15, wherein the milk fat globule membrane is a product obtained by a defatting treatment.

19. The method according to claim 15, wherein the milk fat globule membrane is a product obtained by a treatment with a protease.

20. The method according to claim 15, wherein the milk fat globule membrane is a product obtained from one or more substances selected from the group consisting of milk, cream, buttermilk, whey and processed products thereof.

21. The method according to claim 15, wherein the milk fat globule membrane is orally administered in a daily amount of 0.5 g or more per 60 kg of the patient's body weight.

22. The method according to claim 15, wherein the milk fat globule membrane is orally administered in a daily amount of 1.5 g or more per 60 kg of the patient's body weight.
